(19) United States
(12) Patent Application Publication
(10) Pub. No.: US 2016/0022246 A1
(43) Pub. Date: Jan. 28, 2016

(54) Head Frame with Integrated Pressure Chamber for Non-Invasive Intracranial Pressure Measurements

(71) Applicant: Arminas Ragauskas, Kaunas (LT)

(72) Inventor: Arminas Ragauskas, Kaunas (LT)

(21) Appl. No.: 14/339,982

(22) Filed: Jul. 24, 2014

(51) Int. Cl. A61B 8/00; A61B 8/08; A61B 5/03; A61B 8/06

(52) U.S. Cl. CPC A61B 8/4272; A61B 8/06; A61B 8/488; A61B 5/031; Y10S 901/44

(57) ABSTRACT

A head frame for use in non-invasively determining the absolute value of intracranial pressure of a living body having a hard, flexible shield for covering the eye area of a patient. The shield has an inner surface and an outer transducer surface. An elastic film is sealed along its outer edge to the inner surface of the shield forming an expandable chamber. First and second connectors are located on the shield, permitting an inflow and outflow of pressurized liquid to the chamber. The elastic film expands inward toward the patient with an inflow of pressurized liquid, imparting a pressure against the patient's eye. An adjustable strap is attached to the shield for securing the head frame to the head of the patient.

### FIELD OF THE INVENTION

The present invention generally relates to an apparatus for non-invasively measuring intracranial pressure and more specifically relates to a head frame with an integrated pressure chamber for non-invasively measuring intracranial pressure.

### BACKGROUND OF THE INVENTION

This invention is a new apparatus capable of being used in conjunction with our methods previously described in U.S. Pat. Nos. 5,951,477 and 8,394,025. This new head frame offers advantages over the apparatuses described in the '477 and '025 patents.

An apparatus for determining the pressure and flow inside the ophthalmic artery is described in U.S. Pat. No. 4,907,595 to Strauss. The apparatus uses a rigid chamber that can be affixed and sealed over the human eye so that it can be pressurized to apply an external pressure against the eyeball. An ultrasonic transducer is also mounted to the chamber and oriented to transmit ultrasonic pulses for a Doppler type measurement of the flow inside the ophthalmic artery (OA). The apparatus operates by enabling an operator to increase the pressure to such a level that the blood flow through the OA ceases. The pressure at which this occurs is then an indication of the pressure inside the OA. Typically, the pressure at which this event occurs is in the range of about 170 mmHg.

A problem associated with an apparatus as described in the '595 Patent is that the pressure necessary to obtain the desired measurement is so high that it generally exceeds maximum recommended pressures by a significant amount. When such device is then used for an extended time, tissue damage can arise and may result in an increase in the intracranial pressure, ICP, to unacceptable levels.

In our previously granted '477 and '025 patents we described an apparatus and method for determining the pressure inside the brain. An apparatus as described in the '477 and '025 patents requires annular inflatable pressure chamber with an ultrasonic transducer positioned in the open center of the annular chamber. Thus, the ultrasonic transducer would be positioned against the closed eye-lid of a patient, only separated by the thin, flexible layer of ultrasonically transparent material, sonogel or sonopad.

An apparatus in accordance with either the '477 or the '025 patents restricted the size of the ultrasonic transducer. The diameter of the ultrasonic transducer could only be equal to or less than the diameter of the opening in the annular pressure chamber. The ultrasonic transducer was further limited in size and movement by skull bones around the eye. Further, previously described apparatuses allowed for risk of injury to the patient's eye or facial tissues arround the eye upon movement and adjustment of the ultrasonic transducer's position either manually or robotically.

### SUMMARY OF THE INVENTION

An apparatus in accordance with the invention is a head frame having a lens or shield with a smooth, hard surface and a pressure controlled ultrasonically transparent liquid-filled chamber formed by an elastic film fixed to one side of the lens or shield. Connectors positioned on the lens allow for inlet and outlet of liquid into the pressure chamber. When the head frame of the invention is mounted on the head of a patient and pressure in the chamber is increased, the elastic film expands and conforms to shape of the patient's closed eye imparting a pressure on the tissues around the eye and orbital tissues.

With an apparatus in accordance with the invention one can derive an indication of the pressure inside a skull in a non-invasive manner using previously known methods, such as that described in our '025 patent without risk of injury to the patient's eye. Such methods involve use of an ultrasonic Doppler device to measure blood flow velocities in intracranial and extracranial segments of the ophthalmic artery under varying amounts of pressure applied to the tissues around the eye.

An apparatus in accordance with the invention allows for measurement of the intracranial pressure of a patient without placing the ultrasonic transducer of Doppler device against the eye-lid of the patient. A further aspect of the invention enables the use of a wide range of ultrasonic transducers of different sizes. The diameter of the ultrasonic transducer of Doppler device is not limited, allowing for optimization of the ultrasonic beam and better Doppler signal to noise ratio. Because the ultrasonic transducer of Doppler device is not placed against the eye of the patient, the present invention enables measurement of intracranial pressure by manual operator or robotic driver without discomfort or risk of injury to patient.

An apparatus in accordance with the invention is compatible with all patients and can conform to any patent's eye independent of race and/or facial structure. An apparatus in accordance with the invention provides a surface area that allows for the ultrasonic transducer of Doppler device to be positioned away from the eye and allows for free movement across the entire area of the shield or lens.

It is therefore an object of the invention to allow for the fast and safe optimal positioning of the ultrasonic transducer in order to cross the intracranial and extracranial segments of an ophthalmic artery with the ultrasonic beam at the needed depths from the surface of the ultrasonic transducer and also for non-invasive measurement of intracranial pressure of any patient regardless of age, race, or facial structure.

### BRIEF DESCRIPTION OF THE DRAWINGS

FIG. 1 is a perspective view of the apparatus positioned on the head of a patient.

FIG. 2 is an exploded view of the head frame.

FIGS. 3a-3b are perspective views of the head frame with a collapsed chamber and an expanded chamber, respectively.

FIGS. 4a-4c are front elevational view, top plan view and a side elevational view with the dotted line indicating an expanded chamber, respectively.

FIG. 5 is a right side cutaway view of the head frame positioned on the head of a patient showing the head frame in use with a transducer, the dotted line indicating the elastic film of the expanded chamber.

### DETAILED DESCRIPTION OF THE INVENTION

The exemplary embodiments of the present invention may be further understood with reference to the following description and the related appended drawings, wherein like elements are provided with the same reference numerals. The exemplary embodiments of the present invention are related to a head frame for use in non-invasively determining the absolute value of intracranial pressure (ICP) of a living body.

With an apparatus in accordance with the invention the ICP inside a person's head can be determined from an observation of the blood velocities inside the two segments of the ophthalmic artery (OA) by using an ultrasonic Doppler apparatus which senses the response of the blood flow to a pressure “challenge” applied to the tissues around the eye and orbital tissues. The pressure is applied to the eye at the necessary level for equilibrating parameters representative of the intracranial and extracranial blood flows in the OA leading to the eye. The possibility of this type of measurement has been demonstrated with the analysis presented in our previous U.S. Pat. Nos. 5,951,477 and 8,394,025.

The apparatus of the present invention is significantly improved over previous apparatuses used for noninvasive measurement of ICP for several reasons. The transducer surface of an apparatus in accordance with the present invention is not located against the closed eyelid of a patient, but is located on the shield of the apparatus, reducing risk of injury when an ultrasonic device is being used to take measurements.

Additionally, the shield provides an unrestricted surface to allow for easy movement and manipulation of ultrasonic transducer. Manipulation of the ultrasonic transducer of Doppler device is required to locate and steer the ultrasonic transducer toward the intracranial and extracranial segments of of the ophthalmic artery. The manipulation and steering of the ultrasonic transducer may be performed manually or by robotic steering. Because the transducer is not placed against the eyelid of the patient, robotic manipulation is much safer with the present invention.

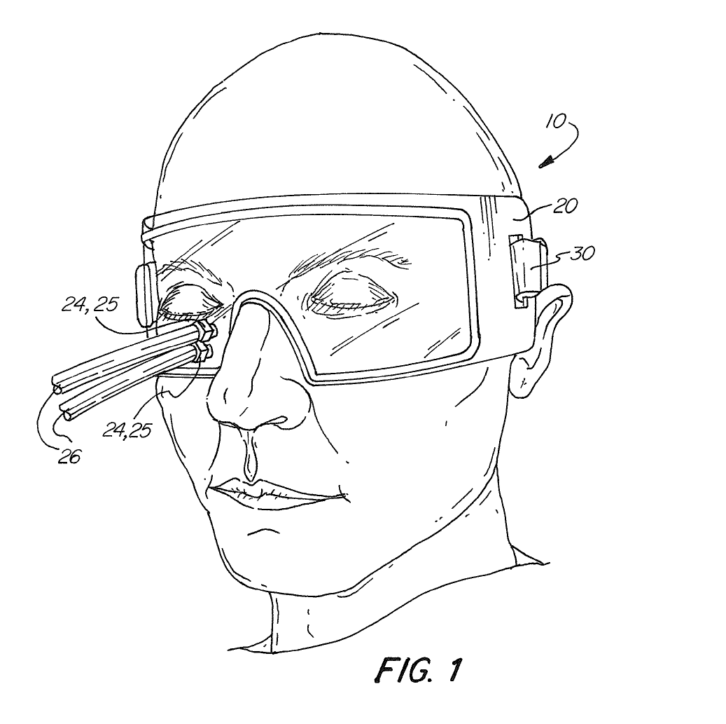

With reference to FIG. 1, a head frame 10 is shown positioned on the head of a patient. Lens or shield 20 is configured to fit over at least an eye area of the patient. Shield 20 in some embodiments extends across the wearer's face from one temple, over the bridge of the nose to the other temple. Shield 20 extends vertically along the wearer's face from about the cheek bone or lower wall of the orbital to about the upper wall of the orbital or the brow of the wearer. In some embodiments the shield 20 extends only over one eye of the patient.

Strap 30 secures head frame 10 in place on the head of the patient. Elastic film 22 is fixed to the inner side of the shield 20. An expandable chamber 28 is formed by the elastic film 22 sealed with the shield 20. To inflate the chamber 28, pressurized liquid is sent into the chamber through a first connector 24. When the chamber 28 is pressurized the elastic film 22 conforms to the eye of a patient and imparts pressure on the eye and tissues around the eye of the patient. Connectors 24, 25 provide an inlet and outlet of liquid into the chamber 28 for adjusting the pressure of the chamber 28 and are in connection with a pressurized liquid. The location of the connectors 24, 25 may be anywhere on the shield that would not interfere with the manipulation of a transducer in measuring or monitoring the ICP of the patient. In the Figures, the connectors 24, 25 are located near the edge of the shield close to the nose of the patient, but could be positioned in other locations.

The connectors 24, 25 are in connection with a source of pressurized liquid. The pressurized liquid may be any ultrasonically transparent liquid. It is preferable that the liquid have low attenuation of ultrasound and provide optimal speed of ultrasound. In some embodiments the pressurized liquid is water. Pure water without gas is the preferred liquid. The pressurized liquid may also be an acoustic gel. Ultrasonically transparent liquid is liquid that does not contain solid particles or gas. It is further preferable that the pressurized liquid does not pose a hazard to the patient in the case of a leak or rupture.

In some embodiments connectors 24, 25 are connected via tubing 26 to a system for monitoring and adjusting the amount of liquid and therefore pressure in the chamber 28. For example, the tubing may be connected to an electromechanical pump, valves, microcontroller with pressure sensor, and other components to monitor and adjust the pressure in the chamber 28.

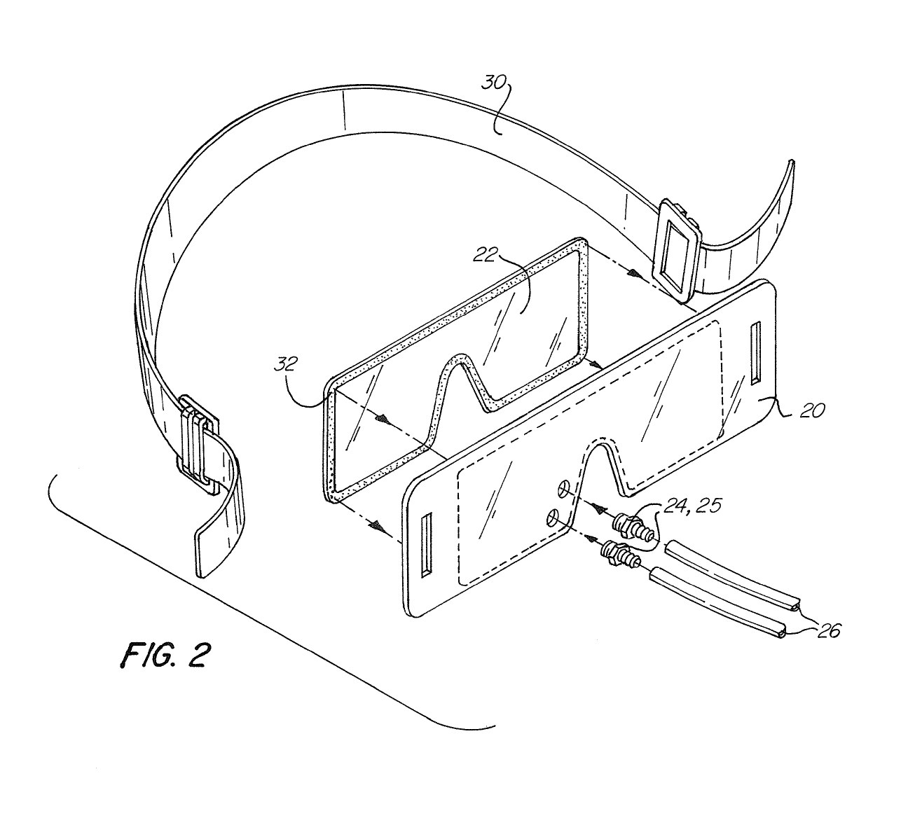

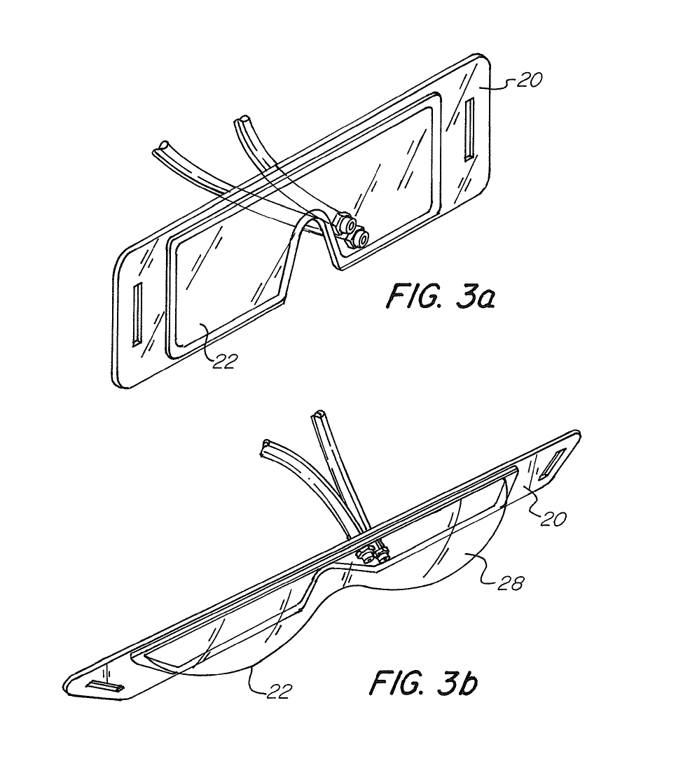

As best seen in FIG. 2, an exploded view of head frame 10 is shown. Shield 20 is configured to cover at least an eye area of a patient. First and second connectors 24, 25 are positioned on shield 20. Elastic film 22 is fixed to shield 20 and expands when pressurized forming a chamber 28 (See FIG. 3a-3b). In some embodiments elastic film 22 is fixed to shield 20 along its outer edge 32 by liquid adhesive. In some embodiments elastic film 22 is fixed to shield 20 in such a way as to form a hermetic seal to prevent liquid from escaping chamber 28 when pressurized up to 100 mmHg.

The strap 30 of the head frame 10 may be elastic or inelastic. The strap 30 is adjustable so as to allow the head frame 10 to be used with any patient. The head frame 10 of this invention may be disposable.

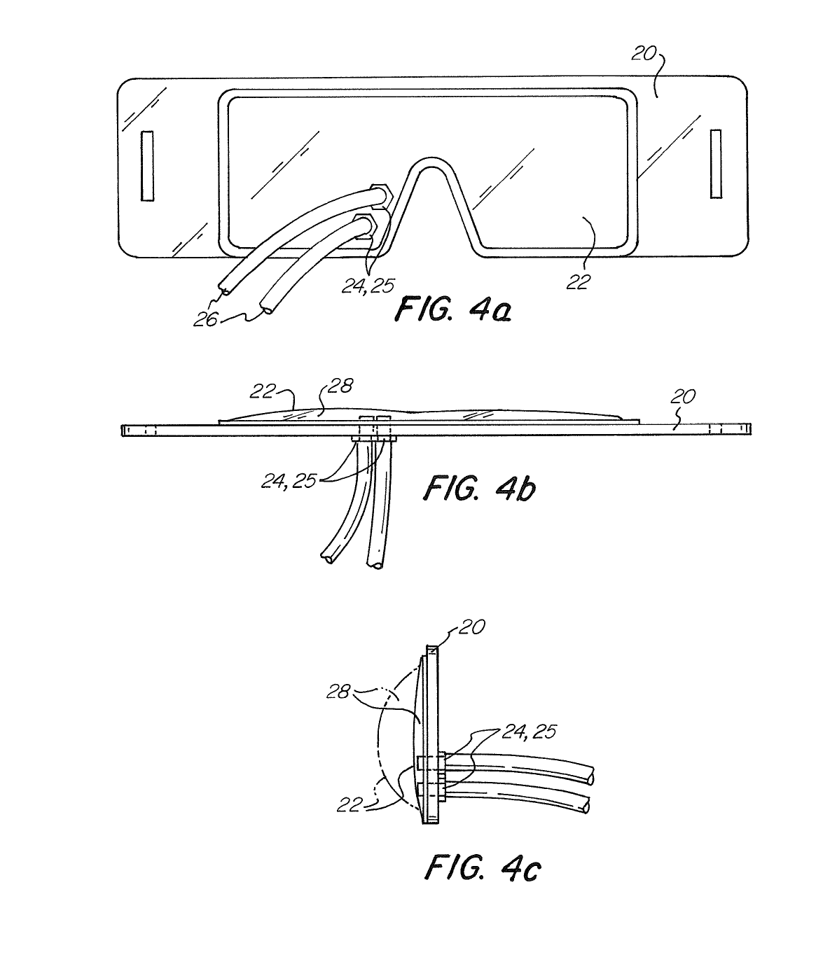

As can be seen best in the series of FIGS. 3 and 4 elastic film 22 flexes and chamber 28 expands with the application of pressure/introduction of liquid. The connectors 24, 25 allow for the inlet and outlet of liquid to and from the chamber 28. The expanded chamber 28 applies pressure to a patient's eye when head frame 10 is positioned on the head of a patient. Elastic film 22 conforms to the shape of the eye when the chamber 28 is pressurized imparting a slight pressurization of the tissues around the eye. These tissues are contiguous with tissues in the posterior orbital portion of the eye socket, so the applied pressure is effective there as well. This results in a pressurization of the extracranial segment of an ophthalmic artery.

It is preferable that in combination, the materials of shield 20 and elastic film 22 must not distort or attenuate an ultrasonic beam in the frequency range 1.5 MHz to 3.0 MHz. It is further preferable that the materials be non-allergenic and transparent. The shield 20 of the current invention provides a suitable surface for making acoustic contact with an ultrasonic transducer 40. In some embodiments shield 20 is made of transparent polycarbonate. The elastic film 22, in some embodiments is made of synthetic polyisoprene latex having a thickness between 40 and 60 microns.

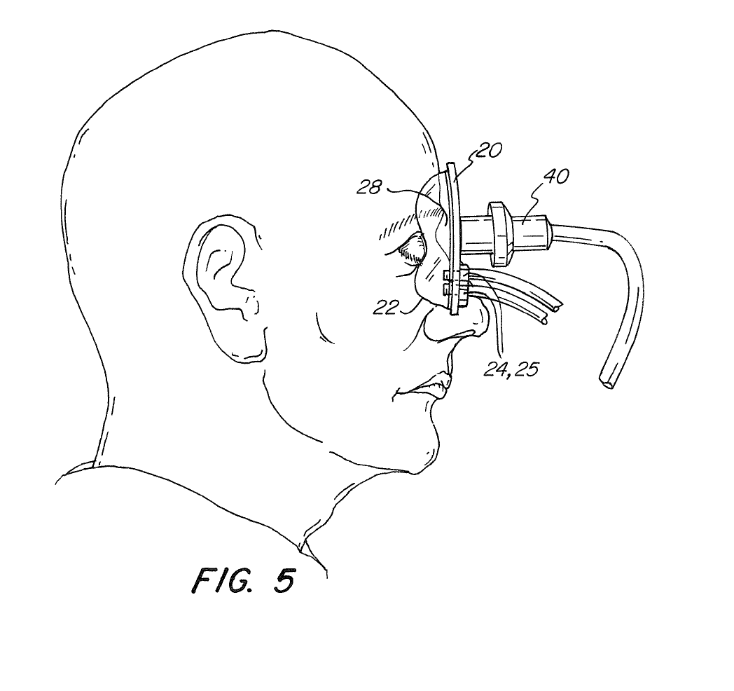

FIG. 5 illustrates the location of the transducer away from the patient's eye. The transducer 40 is manipulated on the transducer surface of the shield 20. FIG. 5 is also the best illustration of the elastic film 22 conforming to the eye of the patient upon inflation of the chamber 28.

An advantage of head frame 10 is that the structure of the head frame 10, with corresponding lens 20 and chamber 28, provides protection for the patient's eye when an ultrasonic transducer of Doppler device or transducer 40 is in use. The apparatus of this invention further allows for safe manual or robotic positioning of ultrasonic transducer and for measurement of intracranial pressure without risk of injury to patient. Head frame 10 allows for accurate measurements by an ultrasonic Doppler device by providing a lens 20 surface that can accommodate a variety of ultrasonic transducer diameters. Ultrasonic transducers used in connection with the invention are preferably between 15 mm and 30 mm in diameter.

By accommodating a wide range of transducer sizes, the apparatus of this invention provides the user with the ability to optimize the diameter of the ultrasonic transducer 40 to achieve the most accurate Doppler signals. Larger diameter transducers provide a greater ability to focus the ultrasonic beam. A focused ultrasonic beam provides stronger signals and better signal to noise ratios.

For use with a patient, the operator or user would place the head frame 10 on the head of the patient with the elastic film 22 proximal the patient's eye area. The operator would adjust the strap 30 to secure the head frame 10 in place. The operator would then apply pressure to the eye of the patient by sending pressurized liquid through the first connector 24. The elastic film 22 would expand and conform to the eye of the patient.

Next, the operator would position the transducer in acoustic contact with the shield 20 for either manual or robotic manipulation. The positions and optimal depths of intracranial and extracranial segments of the ophthalmic artery are located by manipulation of the transducer 40 on the outer surface of the shield 20 away from the patient's eye. The transducer 40 connected to Doppler device can then measure the velocity of the blood flow in the intracranial and extracranial segments of the OA. The operator may adjust the pressure by sending fluid in through connector 24 or allowing fluid out of the chamber 28 through a second connector 25. The adjustment of pressure in the chamber 28 has little or no effect on the angle and position of the ultrasonic transducer 40. This allows the transducer 40 to continue measuring the appropriate blood flow velocities as the pressure is adjusted.

It should be understood that the foregoing is illustrative and not limiting, and that obvious modifications may be made by those skilled in the art without departing from the spirit of the invention. Accordingly, reference should be made primarily to the accompanying claims to determine the scope of the invention.

What is claimed is:

1. A head frame for use in non-invasively determining the absolute value of intracranial pressure of a patient comprising:
a hard, flexible shield adapted to cover the eye area of the patient extending from the right temple over the bridge of the nose to the left temple, and extending from a point below the lower wall of the patient's orbital to a point above the upper wall of the patient's orbital,
wherein the shield further comprises an inner surface and an outer transducer surface;
an elastic film having an outer edge, wherein said elastic film is sealed along its outer edge to the inner surface of the shield forming an expandable chamber;
a first connector located on the shield adapted to permit an inflow of pressurized liquid into said chamber and a second connector located on the shield adapted to permit an outflow of pressurized liquid from the chamber;
wherein said elastic film is adapted to expand inward toward the patient's eye with an inflow of pressurized liquid into to said chamber imparting a pressure against the eye of the patient; and
an adjustable strap attached to the ends of said shield adapted to secure the head frame to the patient.

2. The head frame of claim 1, wherein the elastic film is fixed to the shield by a liquid adhesive.

3. The head frame of claim 1, wherein the chamber is hermetically sealed under pressures up to 100 mmHg.

4. The head frame of claim 1, wherein the outer transducer surface is smooth and suitable for making acoustic contact with an ultrasonic transducer.

5. The head frame of claim 1, wherein the shield is 1.0-2.0 mm polycarbonate.

6. The head frame of claim 1, wherein the elastic film conforms to the outer surface of the patient's closed eye when the chamber is pressurized.

7. A head frame for use in non-invasively determining the absolute value of intracranial pressure of a living body comprising:
a hard, flexible shield for covering the eye area of a patient extending from one temple, over the orbital to the bridge of the nose, and extending from a point below the lower wall of the patient's orbital to a point above the upper wall of the patient's orbital,
wherein the shield further comprises an inner surface and an outer transducer surface;
an elastic film having an outer edge, wherein said elastic film is sealed along its outer edge to the inner surface of the shield forming an expandable chamber;
a first connector located on the shield adapted to permit an inflow of pressurized liquid into said chamber and a second connector located on the shield adapted to permit an outflow of pressurized liquid from the chamber;
wherein said elastic film is adapted to expand inward toward the patient's eye with an inflow of pressurized liquid into to said chamber imparting a pressure against the eye of the patient; and
an adjustable strap attached to the ends of said shield adapted to secure the head frame to the patient.

8. The head frame of claim 7, wherein the elastic film is fixed to the shield by a liquid adhesive.

9. The head frame of claim 7, wherein the chamber is hermetically sealed under pressures up to 100 mmHg.

10. The head frame of claim 7, wherein the outer transducer surface is smooth and suitable for making acoustic contact with ultrasonic transducer.

11. The head frame of claim 7, wherein the shield is 1.0-2.0 mm polycarbonate.

12. The head frame of claim 7, wherein the elastic film conforms to the outer surface of the patient's closed eye when the chamber is pressurized.

13. A system for noninvasively determining the absolute value of intracranial pressure of a patient comprising:
providing a head frame, wherein said head frame comprises a hard flexible shield having an inner surface and an outer transducer surface, an elastic film sealed along an outer edge to the inner surface of the shield forming an expandable chamber, a first and second connector positioned on said shield, and an adjustable strap;
placing said head frame on the patient wherein the elastic film is positioned proximal the patient;
applying external pressure to the eye of the patient by sending pressurized liquid through the first connector inflating said chamber; and
placing an ultrasonic transducer in acoustic contact with said outer surface of said shield to noninvasively locate an intracranial segment and extracranial segment of an ophthalmic artery extending from inside the cranium into the eye by steering the ultrasonic transducer to measure the velocity of blood flow at a plurality of depths through the pressurized chamber.

14. The system of claim 13, wherein the steering of the ultrasonic transducer is performed by robotic means.

15. The system of claim 13, further comprising adjusting the external pressure applied to the eye by sending pressurized liquid through the first connector and permitting the outflow of pressurized liquid from the chamber through the second connector.
